(19) United States
(12) Patent Application Publication
(10) Pub. No.: US 2007/0255228 A1
(43) Pub. Date: Nov. 1, 2007

(54) SHEATH FOR ONE WAY VALVE

(75) Inventor: Michael C. SECONDO, New York, NY (US)

Correspondence Address: KALOW & SPRINGUT LLP, 488 MADISON AVENUE, 19TH FLOOR, NEW YORK, NY, 10022, US

(73) Assignee: Hunter Delivery Systems, Inc., New York, NY (US)

(21) Appl. No.: 11/740,485

(22) Filed: Apr. 26, 2007

Related U.S. Application Data
(60) Provisional application No. 60/795,530, filed on Apr. 27, 2006.

(51) Int. Cl. A61M 5/00

(52) U.S. Cl. 604/246

(57) ABSTRACT

The present invention provides devices and methods for dispensing fluids. By controlling the relationship between valve bodies and elastomeric sheaths that enclose them, one may reduce or eliminate backflow, thereby protecting the integrity of the fluid to be dispensed. Thus, the elastomeric sheath and valve body of the present invention may be used to form a one-way valve for dispensing fluid from a collapsible container.

### CROSS-REFERENCE TO RELATED APPLICATION

This application claims the benefit of U.S. Provisional Application Ser. No. 60/795,530, filed Apr. 27, 2006. The entire disclosure of that application is incorporated by reference as if set forth fully herein.

### FIELD OF THE INVENTION

The present invention relates to a system for dispensing fluids.

### BACKGROUND OF THE PRESENT INVENTION

There is a great need in many industries to dispense fluid products that are susceptible to oxidation and contamination safely. Many products lose their freshness, potency and/or sterility after only a brief period of use. This period or “use life” varies from product to product.

Generally, when fluids are dispensed from a valve, the volume of product delivered from the valve is replaced with an equivalent volume of air. Exposure to this ambient air leads to the entry of oxygen into the container and potentially to contact with contaminants in the air such as microorganisms, atmospheric gases, moisture and dust particles. Consequently, the quality, potency, safety and/or sterility of the remaining product can be compromised by the air and potential contaminants within it. Accordingly, most fluid products are delivered in containers with preservatives.

A small segment of fluid products are delivered in aerosol dispensers and unit dose packaging. Aerosols are limited to high-pressure applications, and they contain highly flammable solvents. Thus, their use life ends in hazardous waste, and their use has long been associated with serious environmental consequences. Unit dose packaging is also problematic; it is the most expensive approach to sterile packaging. Another disadvantage of unit dose packaging is the opportunity for users to save the unpreserved (and no longer sterile) product remaining in the single dose dispenser and to use it on subsequent occasions. This type of noncompliant usage can result in contaminated fluid being dispensed to a patient.

Many industries, including the pharmaceutical, cosmetics, food and beverage industries, depend on the proper preservative ingredients to address product stability, safety and efficacy. However, there are no global standards for preservatives, and there are no ideal preservatives.

One system for maintaining the integrity of fluids to be dispensed is described in the U.S. Pat. No. 6,766,816, issued to Secondo (the “'816 patent”). The '816 patent describes a collapsible dispensing system with a conical one-way valve comprised of a valve body and an elastomeric sheath for dispensing flowable materials where the inside diameter of the sheath is smaller than the outside diameter of the valve body prior to placement over the valve body to form a seal.

However, there is a need for better contamination-safe, propellant-free delivery systems for consumer products that can be easily manufactured and assembled. A delivery system that could extend product use life, improve product performance, reduce the need for harmful preservatives or work in tandem with gentler preservatives would provide a significant public health benefit.

The present invention addresses this need by providing devices and methods for delivering fluid under positive pressure through a one-way valve from a collapsible container so that backflow of air and contaminants into the remaining product in the container is prevented.

### SUMMARY OF THE PRESENT INVENTION

According to one embodiment, the present invention is directed to an improved dispensing system. This dispensing system comprises a sheath that envelops and seals against the outside surface of a conical one-way valve body as in the '816 patent, for propelling and evacuating flowable materials through and out of the valve while preventing backflow of air and contaminants from reaching remaining container content. Also, the sheath is of a smaller diameter than the valve body prior to assembly over and against the outside surface of the valve body. Additionally, the sheath has a greater thickness at the inlet end of the valve than at the outlet end of the valve for providing greater elastic restoring force in evacuating fluid through and out of the valve.

According to another embodiment, the difference in the diameter of the valve body and the smaller diameter sheath is greater at the inlet end than at the outlet end thereby providing greater sealing tension against the outside surface of the valve body at the inlet end of the valve than at the outlet end of the valve.

According to another embodiment, the sheath has a greater thickness at the inlet end than at the outlet end and the difference in the diameter of the valve body and the smaller diameter sheath is greater at the inlet end of the valve than at the outlet end of the valve.

These embodiments provide greater sealing tension and elastic restoring force at the inlet end of the valve than at the outlet end of the valve. Consequently, there is an increased tension of the valve seals and an aid to having fluid flow only in an outward direction. Due to these improved designs, the sheath will collapse and seal against the outside surface of the valve body at the inlet end before sealing at the outlet end upon the reduction of fluid pressure on the inlet port(s). Accordingly, backflow of air or product into the remaining contents of the container is effectively prevented.

The present invention also provides a one-way valve that can be easily manufactured. For example, in some embodiments the present invention provides two and three component valve assemblies that can be produced at a low cost. For example, the present invention can comprise a rigid, vented sleeve laterally enclosing the elastomeric sheath for limiting the radially outward expansion of the sheath and for preventing any build up of air pressure between the sheath and the sleeve. Further, the inside surface of the enclosing sleeve and the corresponding expansion chamber formed between the enclosing sleeve and the elastomeric sheath may be of a roughly conical design. However, even if the inside surface of the enclosing sleeve is roughly conical, the outside surface of the enclosing sleeve need not be conical in shape.

According to another embodiment, the present invention provides a one-way valve with improved ease of assembly. In this embodiment, the present invention has a conical valve body and a conical elastomeric sheath where not only the shape of the components facilitates the lateral enclosure of the sheath along the outside surface of the valve body. The increased elasticity provided by greater sheath wall thickness at the inlet end and/or the reduced interference fit of the sheath at the outlet end further eases assembly of the unit. Although a conical design of the one-way valve is preferred for some uses, in other embodiments, the one-way valve can be of a cylindrical design.

Under another embodiment the present invention provides: a valve assembly for dispensing flowable materials, wherein said valve assembly comprises: (a) a valve body having a longitudinal bore therethrough; and (b) an elastomeric sheath having a longitudinal bore therethrough, wherein said elastomeric sheath has an inlet end and an outlet and said inlet end has a wall thickness and said outlet end has a wall thickness wherein the wall thickness of the inlet end is greater than the wall thickness at the outlet end and wherein said valve body and said elastomeric sheath are conically shaped, and positioned such that said longitudinal bore of said valve body and said longitudinal bore of said elastomeric sheath are coaxial.

Under another embodiment, the present invention provides a valve assembly for dispensing flowable materials, wherein said valve assembly comprises: (a) a valve body having a longitudinal bore therethrough, wherein said valve body has an outer inlet valve body diameter and an outer outlet valve body diameter; and (b) an elastomeric sheath having a longitudinal bore therethrough and the elastomeric sheath has an inner inlet elastomeric sheath diameter and an inner outlet elastomeric sheath diameter, wherein the difference between the outer inlet valve body diameter and the inner inlet elastomeric sheath diameter is greater than the difference between the outer outlet valve body diameter and the outer elastomeric sheath, wherein said valve body and said elastomeric sheath are conically shaped, and positioned such that said longitudinal bore of said valve body and said longitudinal bore of said elastomeric sheath are coaxial

The present invention also provides methods for dispensing fluids using the assemblies of the present invention.

### BRIEF DESCRIPTION OF THE FIGURES

Both the organization and method of operation of the invention, in general, together with further objectives and advantages thereof, may be more easily understood by reference to the figures and the following description. The figures are not intended to limit the scope of this invention, but merely to clarify and to exemplify the invention.

For a more complete understanding of the present invention, reference is now made to the following figures.

FIG. 1 shows a side cross sectional view of a valve assembly according to one embodiment of the present invention.

FIG. 1A shows a side cross sectional view of a valve assembly according to an alternate embodiment of the present invention.

FIG. 1B shows a partial perspective view of a valve assembly according to another alternate embodiment of the present invention.

FIG. 1C shows a partial perspective view of a valve assembly according to another alternate embodiment of the present invention.

FIG. 2 shows a side cross sectional view of a valve assembly according to another alternate embodiment of the present invention.

FIG. 2A shows a side cross sectional view of a valve assembly according to another alternate embodiment of the present invention.

FIG. 2B shows a partial perspective view of the valve assembly of FIG. 2A.

FIG. 3 shows a side cross sectional view of a valve assembly according to another alternate embodiment of the present invention.

FIG. 4 shows a side cross-sectional view of a valve assembly according to another alternate embodiment of the present invention.

FIG. 4A shows a side cross-sectional view of a valve assembly according to another alternate embodiment of the present invention.

FIG. 4B shows a partial perspective view of the valve assembly of FIG. 4A.

FIG. 5 shows a side cross sectional view of a valve assembly according to another alternate embodiment of the present invention.

FIG. 6 shows a side cross sectional view of a valve assembly according to another alternate embodiment of the present invention.

FIG. 7 shows a side cross sectional view of a valve assembly according to another alternate embodiment of the present invention.

FIG. 8 shows a side cross sectional view of the valve assembly according to FIG. 3.

### DETAILED DESCRIPTION

A detailed illustrative embodiment of the present invention is disclosed herein. However, techniques, systems and operating structures in accordance with the present invention may be embodied in a wide variety of forms and modes. Consequently, the specific structural and functional details described herein are merely representative.

Additionally, any of the features of the various embodiments described herein can be used in conjunction with features described in connection with any other embodiments disclosed unless otherwise specified. Accordingly, features described in connection with the various or specific embodiments are not to be construed as not suitable in connection with other embodiments disclosed herein unless such exclusivity is explicitly stated or implicit from the context.

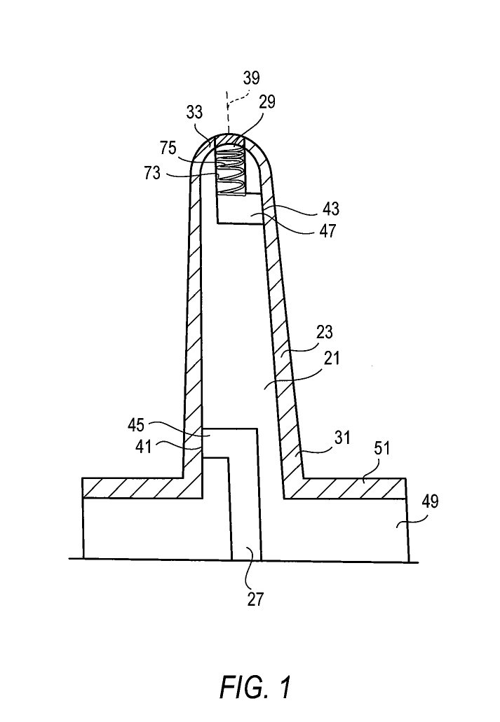

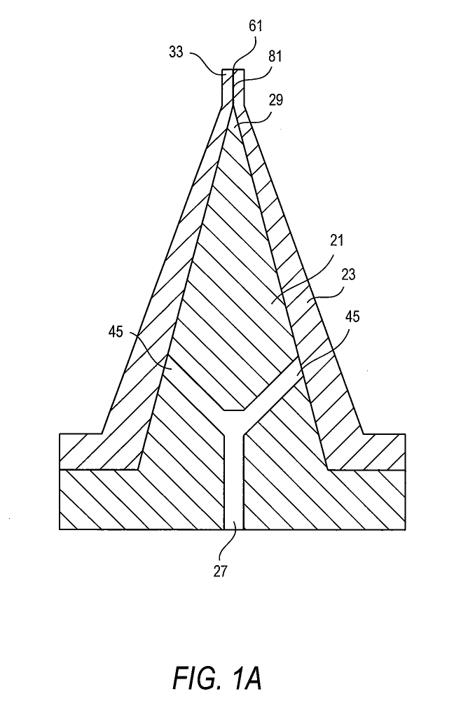

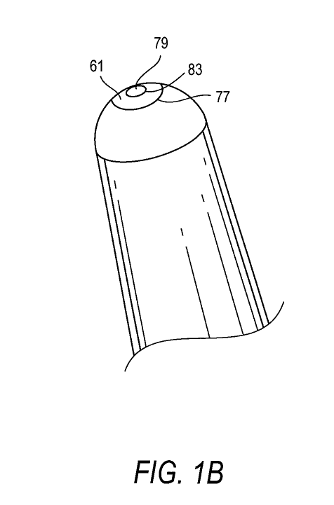

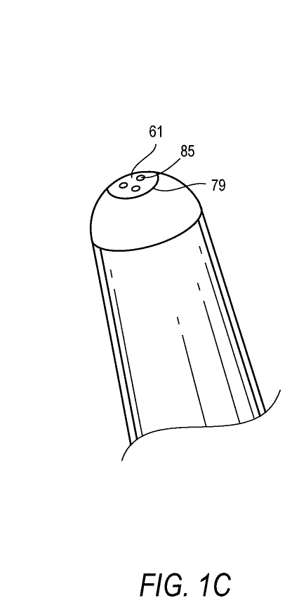

Referring first to FIG. 1, depicted is a side cross sectional view of a valve assembly comprised of a valve body 21 and an elastomeric sheath 23. Both of these parts are conically shaped. Valve body 21 has inlet end 27 and outlet end 29. Elastomeric sheath 23 has inlet end 31 and outlet end 33. Inlet ends 27 and 31 are wider than outlet ends 29 and 33. Valve body 21 and elastomeric sheath 23 have longitudinal bores running along the axis 39 of each part respectively. Inlet ends 27 and 31, as well as corresponding outlet ends 29 and 33 are positioned along axis 39.

Valve body 21 has an inside conical space that tapers inward along the axis from inlet end 27. Valve body 21 has inlet port 41 and outlet port 43 on the side for inlet channel 45 and outlet channel 47. Valve body 21 has an inlet channel 45 toward the inlet end 27 that passes from the inlet conical space to the outside surface of the valve body 21. Valve body 21 also has an outlet channel 47 toward the outlet end 29. Outlet channel 47 transverses the wall of the valve body 21 from the outside surface at an angle to axis 39. Elastomeric sheath 23 also has an inside conical space that tapers inward along the axis from inlet end 31. Elastomeric sheath 23, prior to placement over valve body 21, has an inside diameter that is smaller than the outside diameter of valve body 21 at the inlet end. This difference in diameter is preferably greater at the inlet end than at the outlet end for providing greater sealing tension against the outside surface of the valve body at the inlet end than at the outlet end; and for providing greater fluid ejection ease at the outlet end.

The thickness of elastomeric sheath 23 is preferably greater at the inlet end than at the outlet end for providing greater elastic restoring force against the outside surface of valve body 21 at the inlet end than at the outlet end. Greater elastomer thickness and/or tighter interference fit at the inlet end provides outward sealing direction and pressure forcing fluid through and out of the valve. The interference fit can be greater at both inlet end 27 and outlet end 29 than along the fluid flow path between inlet channel 45 and outlet channel 47 where the interference fit covering inlet port 41 is greater than the interference fit covering outlet port 43 and the fluid flow path in between. Thus, there may be different interference fits at the inlet end, the outlet end and the middle portion (between the inlet and outlet ends) of the assembled valve body, thereby forming an inlet interference fit, an outlet interference fit and a middle interference fit. Accordingly, in some embodiments, when the valve body and elastomeric sheath are assembled, they may form an outlet interference fit, an inlet interference fit and a middle interference fit, wherein the inlet interference fit is greater than the outlet interference fit and said outlet interference fit is greater than the middle interference fit.

A greater interference fit at outlet end 29 axially above the lesser interference fit at outlet port 43 forces fluid through outlet channel 47 and out of the valve. Valve body 21 and elastomeric sheath 23 may also have flange sections 49 and 51 on their respective inlet ends. Flanges 49 and 51 can form a first surface facing in an axial direction toward outlet ends 29 and 33. Valve body flange section 49 and elastomeric sheath flange section 51 can be joined to form a leak-free connection. Outlet channel 47 can have spiral grooves 73 along the axially extending walls to outlet end 29 for supporting and seating antimicrobial component silver coil 75 without impeding fluid flow. The sheath configuration options of a greater wall thickness at the inlet end of the valve than at the outlet end of the valve and/or a greater difference in the diameter of the valve body and the smaller diameter sheath at the inlet end of the valve than at the outlet end of the valve in FIG. 1 can be applied to the embodiments that follow as well. The silver coil component can be configured in embodiments with a discrete outlet channel. The valve body and elastomeric sheath components can also be configured in a cylindrical design.

Referring now to FIGS. 1A-1C, outlet end 29 is configured without a discrete outlet channel inside valve body 21. As shown in FIG. 1A, fluid flows from inlet end 27 passed two inlet channels 45 through an outlet passageway along the exterior surface of valve body 21 and the interior surface of elastomeric sheath 23 to outlet end 29. Elastomeric sheath 23 can form closure 61 in a variety of ways at outlet end 33. In FIG. 1A, closure 61 shows one slit 81, but closure 61 may have a plurality of slits preferably of equal length and radial distance from the axis forming wedge shaped flaps of equal size extending axially over and outward from outlet end 29. The inside surfaces of slits 81 expand to permit fluid flow out of the valve and contract to seal against adjacent and opposite inside surfaces.

In FIG. 1B, the valve body shown has a substantially circular recess 77 around the tip of the outlet end for seating conforming antimicrobial component silver cap 79. Closure 61 has a ring shaped aperture 83 axially at or below the tip of the valve body at the outlet end for sealing partially over and against the outer radial portion of the outside surface of silver cap 79. Fluid exits the valve from between the inside surface of aperture 83 and the outer radial portion of silver cap 79 onto the inner radial portion of silver cap 79.

In FIG. 1C, closure 61 is sealed against the outside surface of silver cap 79. Closure 61 has a plurality of substantially circular orifices 85 positioned over silver cap 79 at the outlet end. Closure 61 expands in response to fluid pressure and fluid exits the valve through orifices 85. Aperture 83, orifice 85 and sealing cap 79 of FIGS. 1A-1C can be designed in a number of shapes and sizes. Closure 61 can be any one of a number of known closures, including but not limited to a duckbill closure, a flattened hollow tube, which when under pressure will expand to permit fluid flow and contract once the pressure is relieved.

The phrase “outer inlet valve body diameter” refers to the diameter of the valve body near the inlet end of the valve body. The phrase “outer outlet valve body diameter” refers to the diameter of the valve body near the outlet end of the valve body. As persons of ordinary skill will appreciate, the walls of the valve body will themselves have a thickness. The term “outer” is used to denoted that the measured diameters include the material that comprises the valve body itself.

The phrase “inner inlet elastomeric sheath diameter” refers to the diameter of the elastomeric sheath's surface that will come into contact with the valve body at the point at which the outer inlet valve body diameter is measured. Similarly, the phrase “inner outlet elastomeric sheath diameter” refers to the diameter of the elastomeric sheath's surface that will come into contact with the valve body at the point at which the outer outlet valve body diameter is measured. In contrast to the term “outer” as used in the preceding paragraph, the term “inner” as used in the aforementioned phrases referring to the elastomeric sheath does not include the material of the sheath itself.

Preferably, the difference between the outer inlet valve body diameter and inner inlet elastomeric sheath diameter is greater than the difference between the outer outlet valve body diameter and inner outlet elastomeric sheath diameter. The measurements are of course taken prior to assembly. When assembled and no fluid is being dispensed, the diameters of the aforementioned sheath regions are essentially the same as the diameters of the corresponding valve body regions.

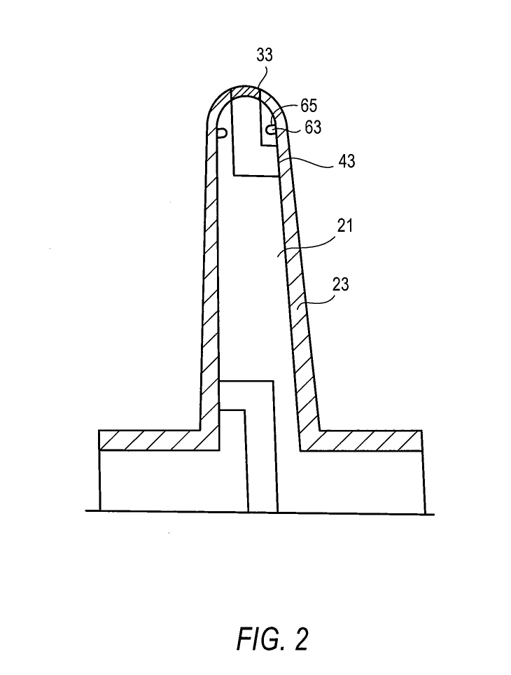

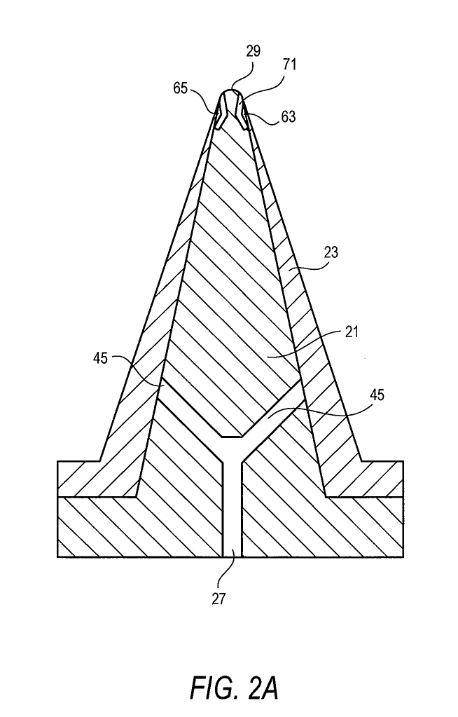

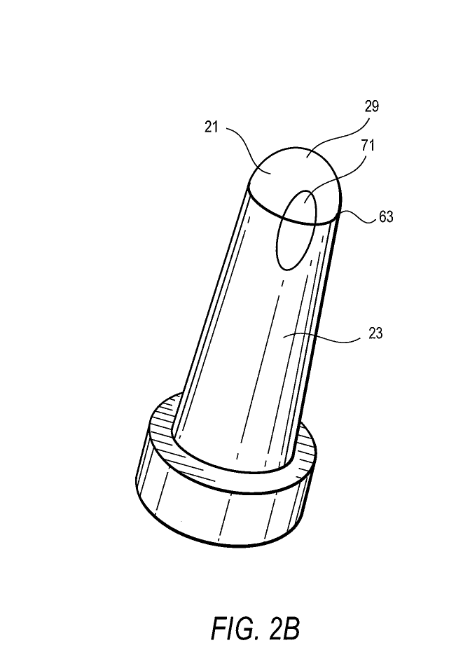

Referring now to FIG. 2, shown is a side cross sectional view of a valve assembly in an alternate embodiment that secures elastomeric sheath 23 in place over and radially around the outside surface of valve body 21 at the tapered end section of elastomeric sheath 23 between outlet port 43 and outlet end 33. Elastomeric sheath 23 has an inward, O-ring like enlargement or protrusion 63 extending circumferentially around the inside surface of elastomeric sheath 23. Valve body 21 has a conforming groove 65 along said circumference for receiving and seating protrusion 63 and for providing attaching means to affix elastomeric sheath 23 to valve body 21. An O-ring can be substituted for protrusion 63.

FIG. 2A shows an example of another alternate embodiment without a discrete outlet channel inside valve body 21. In FIG. 2A, valve body 21 extends axially outward from elastomeric sheath 23 and protrusion 63 at outlet end 29. Valve body 21 has two spoon shaped indentations 71 extending axially under groove 65 to outlet end 29. Fluid flows from inlet end 27 through two inlet channels 45 through an outlet passageway along the exterior surface of valve body 21 and the interior surface of elastomeric sheath 23 into valve body indentations 71 and exits under the line of sealing contact between protrusion 63 and groove 65 through outlet end 29. Indentation 71 can be designed in a variety of shapes to control fluid flow and elastomeric sheath 23 can be designed to conform to, seal over, within or against indentation 71 to drive flowable materials through and out of the valve.

FIG. 2B shows a partial, perspective view of the valve assembly of FIG. 2A. In FIG. 2B, protrusion 63 has a circumferentially extending line of sealing contact against and below the tip of valve body 21 at outlet end 29. Indentation 71 extends axially along the outside surface of valve body under the line of sealing contact and outward from elastomeric sheath 23 at outlet end 29.

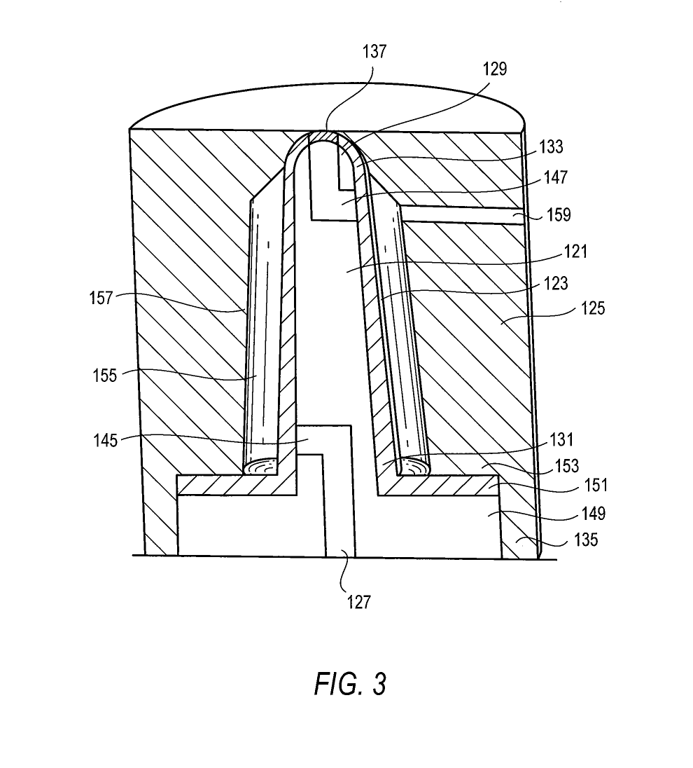

Referring now to FIG. 3, shown is a cross section of one preferred embodiment according to the present invention. Inlet end 127 is the wider end of valve body 121. Inlet end 127 leads to the inside conical space of valve body 121. Valve body 121 has flange 149 on inlet end 127. Inlet channel 145 passes in a generally perpendicular direction to the longitudinal axis of the valve body 121 from the inside conical space of valve body to the outside of valve body. Outlet end 129 is at the narrower end of valve body 121. Outlet channel 147 leads from outlet end 129 for a short distance along the axis passing to the outside of valve body 121. Inlet channel 145 and outlet channel 147 can be modified in number, size, location and design to facilitate the flow of a wide range of fluid viscosities and flow rates. Alternatively, outlet end 129 may be fitted with a drop meter; a hypodermic needle or a nozzle for providing the desired form for the fluid being dispensed including a spray or a stream; or a closure device.

The valve body 121 fits inside of elastomeric sheath 123. Like valve body 121, elastomeric sheath 123 is conically shaped with a flange section 151 at its wider end. Elastomeric sheath 123 also has a tapered end section 133 to match with the tapered end section 129 of valve body 121. Elastomeric sheath 123, prior to placement over valve body 121, has an inside diameter smaller than the outside diameter of valve body 121 at inlet end 131. This difference in the inside diameter of elastomeric sheath 123 to the outside diameter of valve body 121 is preferably greater at inlet end 131 than at outlet end 133. This provides greater sealing tension against the outside surface of the valve body at inlet end 131. The elastomeric sheath 123 then passes more easily over outlet end 129 of valve body 121 before stretching to fit tightly around the inlet end 127 of valve body 121. Preferably elastomeric sheath 123 has a greater thickness at inlet end 131 than at the tapered outlet end section 133. This provides greater elastic restoring force at the inlet end than the outlet end and greater fluid ejection ease through and out of the valve.

Both valve body 121 and elastomeric sheath 123 fit inside enclosing sleeve 125. The inside surface of enclosing sleeve 125 is preferably conical in shape. Enclosing sleeve 125 has a wider inlet end 135 than outlet end 137 positioned along the longitudinal axis. Enclosing sleeve 125 also has a flange section 153 with an increased diameter to contain valve body flange section 149. On the inside of the increased diameter section 153 is a radially inward extending shoulder that secures elastomeric sheath flange section 151. Enclosing sleeve 125 contains expansion chamber 155 in the inside of conical section 157. This space allows elastomeric sheath 123 to expand under fluid pressure coming from inlet channel 145. Enclosing sleeve 125 also features venting means 159 to relieve air pressure against the outside surface of elastomeric sheath 123 within expansion chamber 155. Enclosing sleeve 125 can form a seal with elastomeric sheath 123 and valve body 121 at inlet end 127 and outlet end 129 allowing elastomeric sheath 123 to remain in tight contact with valve body 121. The preferred method of directing fluid flow is through lines of sealing contact with enclosing sleeve 125 along the outside surface of elastomeric sheath 123.

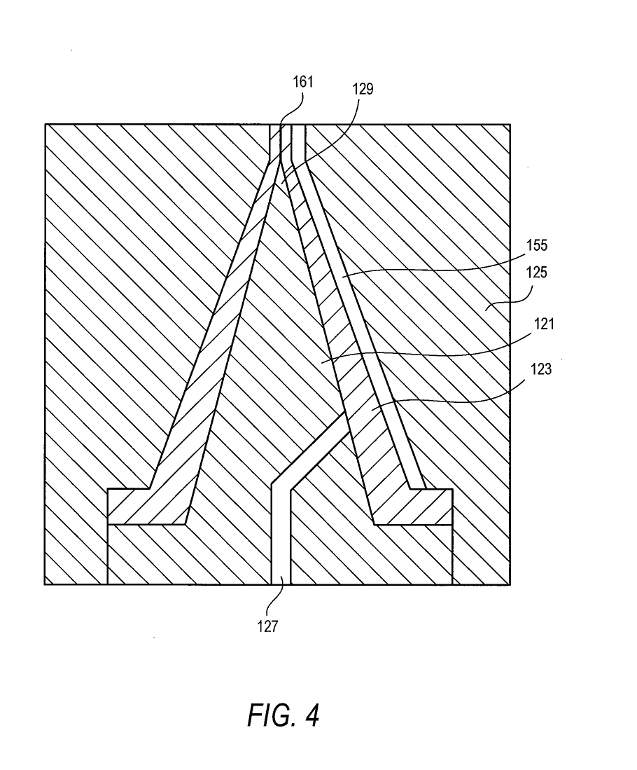

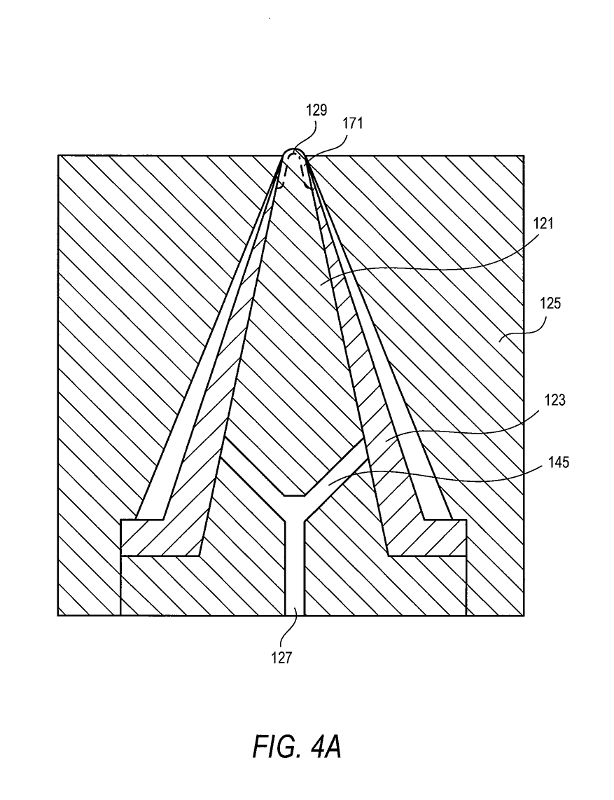

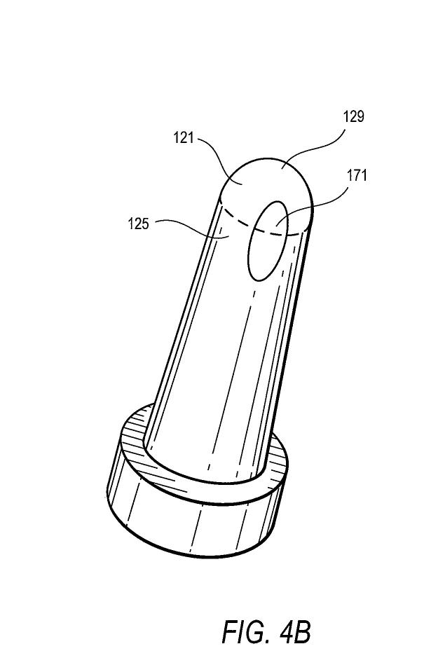

FIG. 4 shows an example of another alternate embodiment of the present invention. In FIG. 4, outlet end 129 is shown without a discrete outlet channel inside valve body 121. Fluid flows from inlet end 127 along the exterior surface of valve body 121 and the interior surface of elastomeric sheath 123 to outlet end 129. Enclosing sleeve 125 provides a line of sealing contact extending in the axial direction against the outside surface of elastomeric sheath 123 that directs the fluid through an outlet passageway along the exterior surface of valve body 121 and elastomeric sheath 123 forms closure 161 for such a passageway. The line of sealing contact limits the area of chamber 155 for the expansion of elastomeric sheath 123 to direct a fluid flow path along, through and out of valve body 121. Closure 161 can extend axially outward from outlet end 129. Closure 161 can be any one of a number of known closures, such as a duckbill closure, a flattened hollow elastomeric tube, which when under pressure will expand to permit fluid flow and contract once the pressure is relieved.

FIG. 4A shows an example of another alternate embodiment without a discrete outlet channel inside valve body 121. In FIG. 4A, valve body 121 extends axially outward from elastomeric sheath 123 and enclosing sleeve 125 at outlet end 129. Enclosing sleeve 125 provides a line of sealing contact at outlet end 129 extending circumferentially around and against elastomeric sheath 123. Valve body 121 has two spoon shaped indentations 171 extending axially from below the line of sealing contact to outlet end 129. Fluid flows from inlet end 127 passed two inlet channels 145 through an outlet passageway along the exterior surface of valve body 121 and the interior surface of elastomeric sheath 123 into valve body indentations 171 and exits under the line of sealing contact between elastomeric sheath 123 and valve body 121 through outlet end 129. Indentation 171 can be designed in a variety of shapes to control fluid flow and elastomeric sheath 123 can be designed to conform to, seal over, within or against indentation 171 to drive flowable materials through and out of the valve.

FIG. 4B shows a partial perspective view of the valve assembly of FIG. 4A. In FIG. 4B, enclosing sleeve 125 has a circumferentially extending line of sealing contact below the tip of valve body 121 at outlet end 129. Indentation 171 extends axially along the outside surface of valve body 121 under the line of sealing contact and outward from enclosing sleeve 125 at outlet end 129 for delivering viscous materials out of the valve.

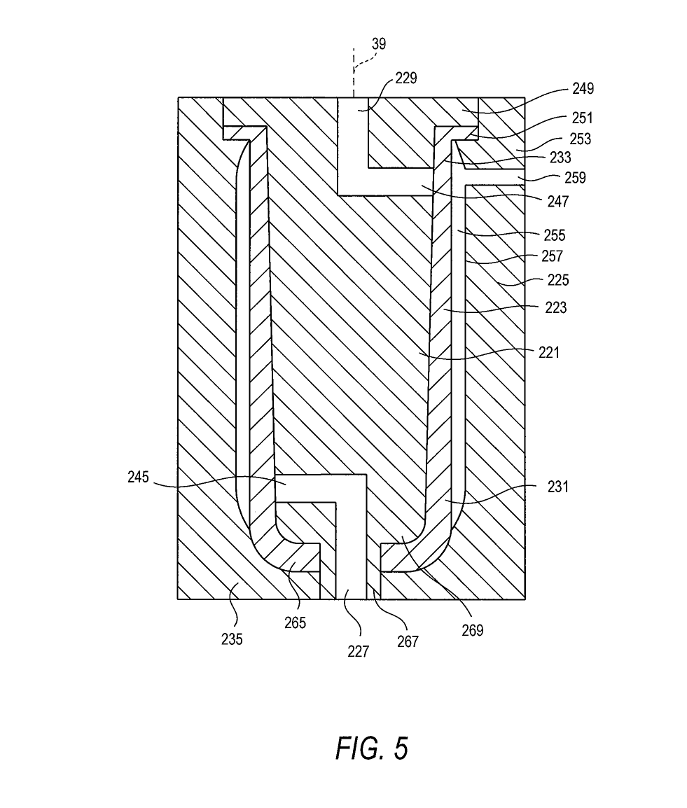

Referring now to FIG. 5, shown is a cross sectional view of another embodiment according to the present invention. Inlet end 227 is the narrower end of conical valve body 221. Outlet end 229 is at the wider end of the valve body. The valve body 221 has tubular section 267 extending axially from inlet end 227. Tapered end section 269 of valve body 221 extends radially outward from tubular section 267 of valve body 221. Valve body 221 has flange 249 on outlet end 229. Inlet end 227 leads to the inside conical space of valve body 221. Inlet channel 245 passes in a generally perpendicular direction to axis 39 of valve body 221 from the inside conical space of valve body 221 to the outside of valve body 221. Outlet channel 247 leads from outlet end 229 for a short distance along the axis passing to the outside of valve body 221.

The valve body 221 fits inside of elastomeric sheath 223. Like valve body 221, elastomeric sheath 223 is conically shaped with a flange section 251 at its wider end. Elastomeric sheath 223 has a tapered end section 231 to match with the tapered end section 269 of valve body 221. Elastomeric sheath 223 also has annular shoulder 265 extending radially inward and against the outside surface of tapered end section 269 of valve body 221 and around tubular section 267 of valve body 221. Elastomeric sheath 223, prior to placement over valve body 221, has an inside diameter smaller than the outside diameter of valve body 221 at inlet end 231. This difference in the inside diameter of elastomeric sheath 223 to the outside diameter of valve body 221 is preferably greater at inlet end 231 than at outlet end 233. This provides greater sealing tension against the outside surface of the valve body at inlet end 231. The elastomeric sheath 223 then passes more easily over inlet end 227 of valve body 221 before stretching to fit tightly around the outlet end 229 of valve body 221. Preferably elastomeric sheath 223 has a greater thickness at inlet end 231 than at the wider outlet end section 233. This provides greater elastic restoring force at the inlet end than the outlet end and greater fluid ejection ease through and out of the valve.

Both valve body 221 and elastomeric sheath 223 fit inside enclosing sleeve 225. The inside surface of enclosing sleeve 225 is preferably conical in shape. Enclosing sleeve 225 has annular shoulder 235 extending radially inward and sealing against the outside surface of conforming annular shoulder 265 of elastomeric sheath 223 and around tubular section 267 of valve body 221. Enclosing sleeve 225 also has a flange section 253 with an increased diameter to contain valve body flange section 249. On the inside of the increased diameter section 253 is a radially inward extending shoulder that secures elastomeric sheath flange section 251. Enclosing sleeve 225 contains expansion chamber 255 in the inside of conical section 257. This space allows elastomeric sheath 223 to expand under fluid pressure coming from inlet channel 245. Enclosing sleeve 225 also features venting means 259 to relieve air pressure against the outside surface of elastomeric sheath 223 within expansion chamber 255. Enclosing sleeve 225 can form a seal with elastomeric sheath 223 and valve body 221 at inlet end 227 and outlet end 229 allowing elastomeric sheath 223 to remain in tight contact with valve body 221. The preferred method of directing fluid flow is through lines of sealing contact with enclosing sleeve 225 along the outside surface of elastomeric sheath 223.

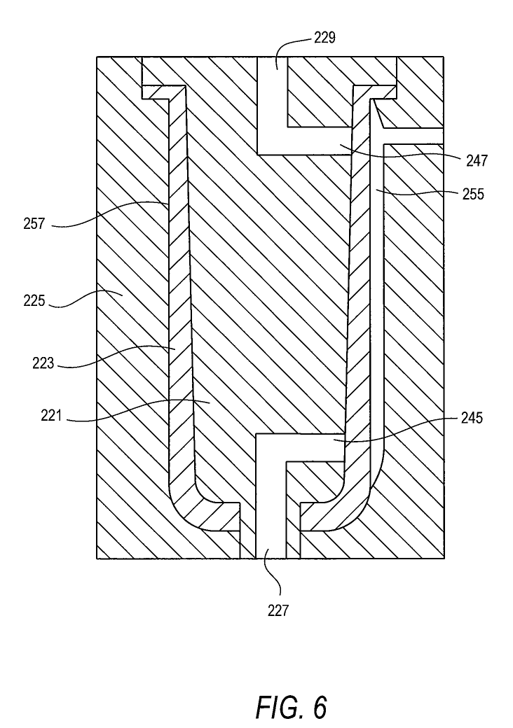

FIG. 6 shows an example in another embodiment of the present invention. Inlet channel 245 and outlet channel 247 are aligned on the same side of valve body 221 to shorten the fluid flow path. Inside conical section 257 of enclosing sleeve 225 has a line of sealing contact against the outside surface of elastomeric sheath 223 extending in the axial direction from inlet end 227 to outlet end 229 180° opposite inlet channel 245 and outlet channel 247 preventing a spiral fluid flow path along the outside surface of valve body 221. The line of sealing contact limits the area of chamber 255 for the expansion of elastomeric sheath 223 to provide a direct fluid flow path along, through and out of valve body 221.

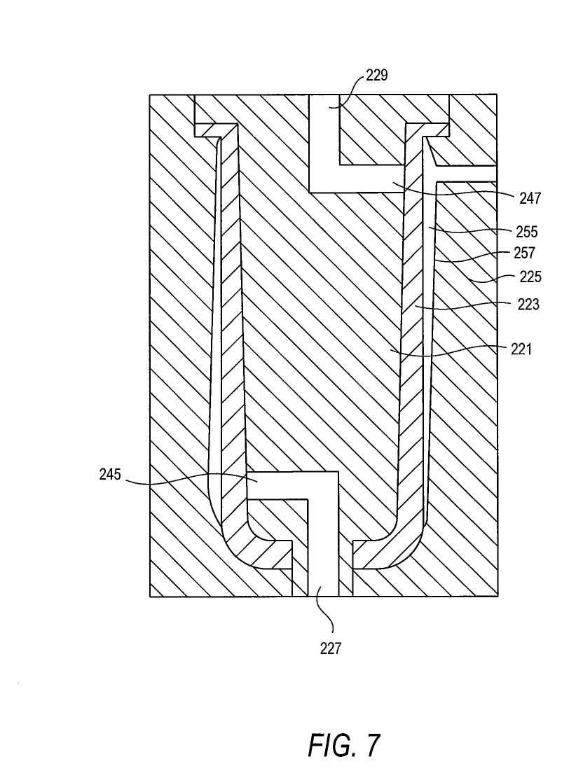

FIG. 7 shows an example in another embodiment of the present invention. Inlet channel 245 and outlet channel 247 are aligned on opposite sides of valve body 221 to lengthen the fluid flow path shown in FIG. 6. Inside conical section 257 of enclosing sleeve 225 has a line of sealing contact against the outside surface of elastomeric sheath 223 extending diagonally across valve body 221 in the axial direction from inlet end 227 to outlet end 229 preventing a spiral fluid flow path along the outside surface of valve body 221. The line of sealing contact limits the area of chamber 255 for the expansion of elastomeric sheath 223 to provide a direct fluid flow path along, through and out of valve body 221.

Inlet channel 245 and outlet channel 247 can be modified in number, size, location and design to facilitate the flow of a wide range of fluid viscosities and flow rates. Delivery of more viscous fluid at higher flow rates can be produced by increasing the number and/or the diameter of inlet and outlet channels. The location of inlet and outlet channels can also be used to facilitate flow rate. For example, the aligned port configuration of inlet channel 245 and outlet channel 247 shown in FIG. 6 has a shorter, more direct fluid flow path preferred for delivery of more viscous fluids than the 180° opposed port configuration shown in FIG. 7.

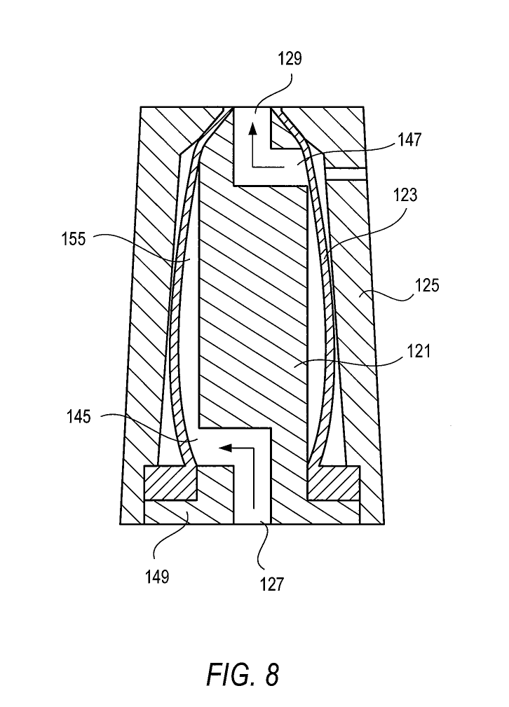

Referring now to FIG. 8 and to the corresponding features in FIG. 5, enclosing sleeve 125 can provide protective cover and resistance against forces from outside inlet channel 145 and outlet channel 147 and therefore ensure that flowable materials pass through and out of the valve without backflow of air and contaminants into the container. Enclosing sleeve 125 can be attached to valve body flange section 149 via any attaching means, including but not limited to, snap fitting, press fitting, heat sealing or welding. Any other method for joining parts to obtain a leak free connection may be used.

Valve body 121, elastomeric sheath 123 and enclosing sleeve 125 are assembled to make a conical one-way valve. The valve is then attached to the collapsible container to form the complete fluid dispensing system. Fluid flows from the container into inlet end 127 and passes into the conical space of valve body 121. As more fluid enters this space, the pressure increases and fluid is forced through inlet channel 145. Once the pressure is sufficient, elastomeric sheath 123 deforms and allows fluid in to expansion chamber 155. As expansion chamber 155 fills, the fluid flows along the outside surface of valve body 121 before it passes back into valve body 121 through outlet channel 147 and finally out of the valve through outlet end 129. Reduction of fluid pressure from inlet end 127 sufficient to enable elastomeric sheath to collapse and reseal first against the outside surface of inlet channel 145, provides the outward sealing direction and force against the outside surface of valve body 121 to drive all fluid within expansion chamber 155 out of the valve through outlet channel 147 and outlet end 129. Backflow and therefore contamination is prevented by the greater sealing tension and elastic restoring force of elastomeric sheath 123 at the inlet end than at the outlet end.

All three components, namely valve body 121, elastomeric sheath 123 and enclosing sleeve 125 may contain and/or be coated with an anti-microbial agent to prevent contamination of sterile fluids. An inert, non-elutable anti-microbial agent is preferred. Likewise, all three components may be composed of materials that are stable to solutions under a broad pH range and resistant to degradation under exposure to a wide range of organic and aqueous solvents.

Within ranges of interference fit, increased sealing tension may be expected with greater differences in valve body diameter to undersized sheaths. Sheaths with a greater wall thickness may be expected to provide more elastic restoring force. Sheaths with a smaller wall thickness may be expected to provide greater ejection ease. Preferably the wall thickness of the sheath is in the range of 8/1000″ to 35/1000″. The durometer is preferably in the range of 25-60 (A). The wall thickness and durometer of the sheath can be adjusted for optimal sealing and ejection ease. A wall thickness ratio of at least 2 to 1 from inlet to outlet is preferred where the difference in diameter of the valve body and the smaller diameter sheath is not reduced from inlet end to outlet end. The preferred range of sheath inner diameters comprising the interference fit is 0.5 to 0.9 times the outer diameter of the valve body. The most preferred sheath inner diameter comprising the interference fit is approximately 0.75 times (0.70-0.80 times) the outer diameter of the valve body at the inlet end.

Tapered sheath wall thickness and/or reduced interference fit is configured to provide greater sheath compression at the inlet end than at the outlet end, as well as to overcome pressure gradient force, which is apt to be greater at the narrower end in a conical valve design. Inlet and outlet ports and channels can be aligned, staggered and configured in number, placement and size to achieve desired fluid flow paths and flow rates, as well as to enhance sealing effectiveness and facilitate the prevention of fluid backflow. Inlet port(s) and outlet port(s)/passageway(s) may be configured at a predetermined fluid flow path distance for effective sealing of the valve and prevention of backflow. The length of the flow path between inlet port(s) and outlet port(s)/passageway(s) is preferably at least one-half inch, and in some embodiments preferably 2 to 2.5 times the length of the diameter of the valve body at the wider end. The length of the flow path can be equal to the wider valve body diameter in shorter, low profile valves when configured with greater sheath wall thicknesses and larger diameter ports. Low profile valves can provide the same effective sealing capabilities, backflow prevention and fluid ejection ease as a longer valve with a thinner elastomeric sheath.

Appropriate valve materials may depend on the nature of the fluid and the application. Preferable materials for the valve body and elastomeric sheath have low absorbance, high adhesive surface characteristics that form bonds that maintain a quick and firm sealing tension. Preferred materials for the elastomeric sheath are silicone, polystyrene butadiene, rubber latex and butyl rubber. Preferred materials for the valve body are polymethacrylate and polysulfone. Elastomeric materials with a durometer of 70 (A) or higher may be used as well. Also, Teflon valve bodies can be made effective sealing agents when configured with greater sheath wall thicknesses although the material does not have a preferred sealing surface. Some materials with low adhesive surface characteristics can be treated to increase adhesive bonds to elastomeric materials. For example, polypropylene can be heat treated to enhance sealing effectiveness in applications requiring solvent resistance.

Previous inventions in this area have been used to protect chemicals, medicines, personal hygiene products and other flowable materials susceptible to contaminations by atmospheric gases and microorganisms. The designs of the various embodiments of the present invention, with their enhanced ease of manufacturing and assembly will decrease the cost of current applications of one way valves and extend the use of one way valves to previously prohibited applications. For example, in embodiments that contain a conical shape of the valve in a 180° opposed port configuration, the system allows the fluid to flow through the valve in a spiral fashion along a path that can be greater than the length of the valve body. This enables the valve body to be smaller, cheaper and easier to assemble without functional sacrifice.

While the present invention has been described with reference to one or more preferred embodiments, such embodiments are merely exemplary and are not intended to be limiting or represent an exhaustive enumeration of all aspects of the invention. The scope of the invention, therefore, shall be defined solely by claims not provided herein. Further, it will be apparent to those skilled in the art that numerous changes may be made in such details without departing from the spirit and the principles of the invention. It should be appreciated that the present invention is capable of being embodied in cylindrically shaped, wedge shaped or other forms without departing from its essential characteristics.

I claim:

1. A valve assembly for dispensing flowable materials, wherein said valve assembly comprises: (a) a valve body having a longitudinal bore therethrough; and (b) an elastomeric sheath having a longitudinal bore therethrough, wherein said elastomeric sheath has an inlet end and an outlet and said inlet end has a wall thickness and said outlet end has a wall thickness wherein the wall thickness at the inlet end is greater than the wall thickness at the outlet end and wherein said valve body and said elastomeric sheath are conically shaped, and positioned such that said longitudinal bore of said valve body and said longitudinal bore of said elastomeric sheath are coaxial.

2. The valve assembly of claim 1, wherein the wall thickness of said inlet end is at least twice as large as the wall thickness of said outlet end.

3. A valve assembly for dispensing flowable materials, wherein said valve assembly comprises: (a) a valve body having a longitudinal bore therethrough, wherein said valve body has an outer inlet valve body diameter and an outer outlet valve body diameter; and (b) an elastomeric sheath having a longitudinal bore therethrough and the elastomeric sheath has an inner inlet elastomeric sheath diameter and an inner outlet elastomeric sheath diameter, wherein the difference between the outer inlet valve body diameter and the inner inlet elastomeric sheath diameter is greater than the difference between the outer outlet valve body diameter and the outer outlet elastomeric sheath diameter,
wherein said valve body and said elastomeric sheath are conically shaped, and positioned such that said longitudinal bore of said valve body and said longitudinal bore of said elastomeric sheath are coaxial.

4. The valve assembly of claim 3, wherein the inner inlet elastomeric sheath diameter and the inner outlet elastomeric sheath diameter are each 0.5 to 0.9 times the outer diameter of the valve body when measured in the unassembled state.

5. The valve assembly of claim 4, wherein said elastomeric sheath has an inlet end and an outlet end and said inlet end has a wall thickness and said outlet end has a wall thickness, wherein the wall thickness at the inlet end is greater than the wall thickness at the outlet end.

6. The valve assembly according to claim 1 further comprising and enclosing sleeve.

7. The value assembly according to claim 3 further comprising an enclosing sleeve.

8. The valve assembly of claim 5, wherein the wall thickness of said inlet end is at least twice as large as the wall thickness of said outlet end.

9. The valve assembly of claim 3, wherein when said valve body and said elastomeric sheath are assembled said valve body and said elastomeric sheath form an outlet interference fit, an inlet interference fit and a middle interference fit, wherein said inlet interference fit is greater than said outlet interference fit and said outlet interference fit is greater than said middle interference fit.

10. The valve assembly of claim 3, wherein when said valve body and said elastomeric sheath are assembled said valve body and said elastomeric sheath form an outlet interference fit, an inlet interference fit and a middle interference fit, wherein said inlet interference fit and said outlet interference fit are greater than said middle interference fit.

11. The valve assembly of claim 1, wherein said valve body has a valve body outlet end that has a substantially circular recess.

12. The valve assembly of claim 11 further comprising an antimicrobial silver cap.

13. The valve assembly of claim 3, wherein said valve body has a valve body outlet end that has a substantially circular recess.

14. The valve assembly of claim 13 further comprising an antimicrobial silver cap.

15. The valve assembly of claim 12 further comprising a closure element, wherein said closure element has a plurality of substantially circular orifices that are positioned over said silver cap.

16. The valve assembly of claim 14 further comprising a closure element, wherein said closure element has a plurality of substantially circular orifices that are positioned over said silver cap.

17. The valve assembly of claim 1, wherein said valve body further comprises an inlet channel and an outlet channel, wherein said inlet channel and said outlet channel are aligned on the same side of said valve body.

18. The valve assembly of claim 3, wherein said valve body further comprises an inlet channel and an outlet channel, wherein said inlet channel and said outlet channel are aligned on the same side of said valve body.

19. The valve assembly of claim 1, wherein said valve body further comprises an inlet channel and an outlet channel, wherein said inlet channel and said outlet channel are aligned on opposite sides of said valve body.

20. The valve assembly of claim 3, wherein said valve body further comprises an inlet channel and an outlet channel, wherein said inlet channel and said outlet channel are aligned on opposite sides of said valve body.

21. A valve assembly for dispensing flowable materials, wherein said valve assembly comprises: (a) a valve body having a longitudinal bore therethrough; and (b) an elastomeric sheath having a longitudinal bore therethrough, wherein said elastomeric sheath has an inlet end and an outlet and said inlet end has a wall thickness and said outlet end has a wall thickness wherein the wall thickness at the inlet end is greater than the wall thickness at the outlet end and wherein said valve body and said elastomeric sheath are cylindrically shaped and positioned such that said longitudinal bore of said valve body and said longitudinal bore of said elastomeric sheath are coaxial.

22. The valve assembly of claim 21, wherein the wall thickness of said inlet end is at least twice as large as the wall thickness of said outlet end.

23. A valve assembly for dispensing flowable materials, wherein said valve assembly comprises: (a) a valve body having a longitudinal bore therethrough, wherein said valve body has an outer inlet valve body diameter and an outer outlet valve body diameter; and (b) an elastomeric sheath having a longitudinal bore therethrough and the elastomeric sheath has an inner inlet elastomeric sheath diameter and an inner outlet elastomeric sheath diameter, wherein the difference between the outer inlet valve body diameter and the inner inlet elastomeric sheath diameter is greater than the difference between the outer outlet valve body diameter and the outer outlet elastomeric sheath diameter,
wherein said valve body and said elastomeric sheath are cylindrically shaped, and positioned such that said longitudinal bore of said valve body and said longitudinal bore of said elastomeric sheath are coaxial.

24. The valve assembly of claim 23, wherein the inner inlet elastomeric sheath diameter and the inner outlet elastomeric sheath diameter are each 0.5 to 0.9 times the outer diameter of the valve body when measured in the unassembled state.

25. The valve assembly of claim 24, wherein said elastomeric sheath has an inlet end and an outlet end and said inlet end has a wall thickness and said outlet end has a wall thickness, wherein the wall thickness at the inlet end is greater than the wall thickness at the outlet end.

26. The valve assembly according to claim 21 further comprising an enclosing sleeve.

27. The value assembly according to claim 23 further comprising an enclosing sleeve.

28. The valve assembly of claim 25, wherein the wall thickness of said inlet end is at least twice as large as the wall thickness of said outlet end.

29. The valve assembly of claim 23, wherein when said valve body and said elastomeric sheath are assembled said valve body and said elastomeric sheath form an outlet interference fit, an inlet interference fit and a middle interference fit, wherein said inlet interference fit is greater than said outlet interference fit and said outlet interference fit is greater than said middle interference fit.

30. The valve assembly of claim 23, wherein when said valve body and said elastomeric sheath are assembled said valve body and said elastomeric sheath form an outlet interference fit, an inlet interference fit and a middle interference fit, wherein said inlet interference fit and said outlet interference fit are greater than said middle interference fit.

31. The valve assembly of claim 21, wherein said valve body has a valve body outlet end that has a substantially circular recess.

32. The valve assembly of claim 31 further comprising an antimicrobial silver cap.

33. The valve assembly of claim 23, wherein said valve body has a valve body outlet end that has a substantially circular recess.

34. The valve assembly of claim 33 further comprising an antimicrobial silver cap.

35. The valve assembly of claim 32 further comprising a closure element, wherein said closure element has a plurality of substantially circular orifices that are positioned over said silver cap.

36. The valve assembly of claim 34 further comprising a closure element, wherein said closure element has a plurality of substantially circular orifices that are positioned over said silver cap.

37. The valve assembly of claim 21, wherein said valve body further comprises an inlet channel and an outlet channel, wherein said inlet channel and said outlet channel are aligned on the same side of said valve body.

38. The valve assembly of claim 23, wherein said valve body further comprises an inlet channel and an outlet channel, wherein said inlet channel and said outlet channel are aligned on the same side of said valve body.

39. The valve assembly of claim 21, wherein said valve body further comprises an inlet channel and an outlet channel, wherein said inlet channel and said outlet channel are aligned on opposite sides of said valve body.

40. The valve assembly of claim 23, wherein said valve body further comprises an inlet channel and an outlet channel, wherein said inlet channel and said outlet channel are aligned on opposite sides of said valve body.
